(19) United States
(12) Patent Application Publication
(10) Pub. No.: US 2026/0263036 A1
(43) Pub. Date: Sep. 10, 2026

(54) ULTRASOUND ENDOSCOPE SYSTEM

(71) Applicant: FUJIFILM Corporation, Tokyo (JP)

(72) Inventor: Yasuhiko MORIMOTO, Ashigarakami-gun (JP)

(73) Assignee: FUJIFILM Corporation, Tokyo (JP)

(21) Appl. No.: 19/549,005

(22) Filed: Feb. 25, 2026

(30) Foreign Application Priority Data
Mar. 5, 2025 (JP) 2025-034436

(51) Int. Cl. A61B 8/12

(52) U.S. Cl. CPC A61B 8/12

(57) ABSTRACT

An ultrasound endoscope system includes: an insertion part; plural transducers; an observation window; an imaging element; a treatment tool outlet port; an elevator that is provided at least partially between the plural transducers and the observation window in a longitudinal direction of the insertion part; and a processor that acquires the image signal output from the imaging element, as defined herein, the processor is configured to display a first optical image generated from the image signal on a display device in a case where the observation window is in air, and display a second optical image generated from the image signal on the display device in a case where the observation window is in water, at least a part of the elevator is included in the first optical image, and the elevator is not included in the second optical image.

### CROSS-REFERENCE TO RELATED APPLICATION

The present application claims priority under 35 U.S.C. § 119 to Japanese Patent Application No. 2025-034436 filed on Mar. 5, 2025. The above application is hereby expressly incorporated by reference, in its entirety, into the present application.

### BACKGROUND OF THE INVENTION

### 1. Field of the Invention

The present disclosed technology relates to an ultrasound endoscope system.

### 2. Description of the Related Art

JP2021-7805A describes a convex type ultrasonic endoscope.

### SUMMARY OF THE INVENTION

An object of the present disclosure is to improve operability of an unltrasonic endoscope.

An ultrasound endoscope system according to one embodiment of the disclosed technology includes an insertion part that is inserted into a subject; a plurality of transducers that are provided on a distal end side of the insertion part and that transmit and receive ultrasound waves; an observation window that is provided on a proximal end side of the plurality of transducers in the insertion part; an imaging element that images the subject through the observation window and outputs an image signal of the subject; a treatment tool outlet port that is provided on the distal end side of the insertion part and that is an outlet port for a treatment tool; an elevator that is provided at least partially between the plurality of transducers and the observation window in a longitudinal direction of the insertion part and that is able to change an outlet direction of the treatment tool led out from the outlet port; and a processor that acquires the image signal output from the imaging element, in which the processor is configured to display a first optical image generated from the image signal on a display device in a case where the observation window is in air, and display a second optical image generated from the image signal on the display device in a case where the observation window is in water, at least a part of the elevator is included in the first optical image, and the elevator is not included in the second optical image.

According to the disclosed technology, the operability of the ultrasound endoscope can be improved.

### BRIEF DESCRIPTION OF THE DRAWINGS

FIG. 1 is a schematic configuration diagram showing an example of an ultrasound endoscope system 100 using an ultrasonic endoscope 1 according to an embodiment of the technology of the present disclosure.

FIG. 2 is a perspective view showing an appearance of a distal end rigid portion 34.

FIG. 3 is a plan view (top view) of the distal end rigid portion 34.

FIG. 4 is a side cross-sectional view of the distal end rigid portion 34.

FIG. 5 is a diagram showing an example of a first optical image IMA displayed on a monitor 120.

FIG. 6 is a diagram showing an example of a second optical image IMB displayed on the monitor 120.

FIG. 7 is a plan view showing a modification example of the distal end rigid portion 34.

FIG. 8 is a side cross-sectional view of the distal end rigid portion 34 shown in FIG. 7.

FIG. 9 is a diagram showing an example of a first optical image IMC displayed on the monitor 120.

FIG. 10 is a diagram showing an example of a second optical image IMD displayed on the monitor 120.

FIG. 11 is a diagram showing an example of an imaging range in water.

### DESCRIPTION OF THE PREFERRED EMBODIMENTS

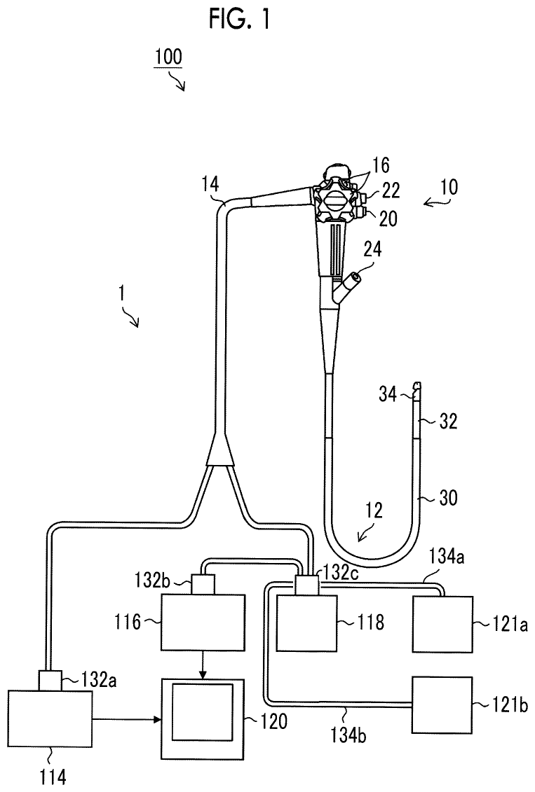

FIG. 1 is a schematic configuration diagram showing an example of an ultrasound endoscope system 100 using an ultrasonic endoscope 1 according to an embodiment of the technology of the present disclosure. The ultrasound endoscope system 100 comprises the ultrasonic endoscope 1, an ultrasound processor device 114 that generates an ultrasound image, an endoscope processor device 116 that generates an endoscopic image, a light source device 118 that supplies illumination light for illuminating an inside of a body cavity to the ultrasonic endoscope 1, a monitor 120 constituting a display device that displays the ultrasound image and an optical image, a water supply tank 121a that stores cleaning water and the like, and a suction pump 121b that aspirates an object to be suctioned in the body cavity.

The ultrasonic endoscope 1 has an insertion part 12 that is inserted into a body cavity of a subject, an operating part 10 that is continuously provided in a proximal end part of the insertion part 12 and that is used by an operator to perform an operation, and a universal cord 14 that has one end connected to the operating part 10.

In the operating part 10, an air and water supply button 22 that opens and closes an air and water supply pipe line (not shown) from the water supply tank 121a, and a suction button 20 that opens and closes a suction pipe line (not shown) from the suction pump 121b are provided side by side. In the operating part 10, a pair of angle knobs 16 and a treatment tool inlet port 24 are provided.

At the other end of the universal cord 14, an ultrasound connector 132a that is connected to the ultrasound processor device 114, an endoscope connector 132b that is connected to the endoscope processor device 116, and a light source connector 132c that is connected to the light source device 118 are provided. The ultrasonic endoscope 1 is attachably and detachably connected to the ultrasound processor device 114, the endoscope processor device 116, and the light source device 118 via the connectors 132a, 132b, and 132c, respectively. The connector 132c comprises an air and water supply tube 134a that is connected to the water supply tank 121a, and a suction tube 134b that is connected to the suction pump 121b.

The insertion part 12 includes, in order from a distal end side, a distal end rigid portion 34, a bendable part 32 that is connected to a proximal end side of the distal end rigid portion 34, and a soft part 30 that connects the proximal end side of the bendable part 32 and the distal end side of the operating part 10.

The bendable part 32 is remotely operated to be bent by rotatably operating the pair of angle knobs 16 provided in the operating part 10. With this, the distal end rigid portion 34 can be directed in a desired direction.

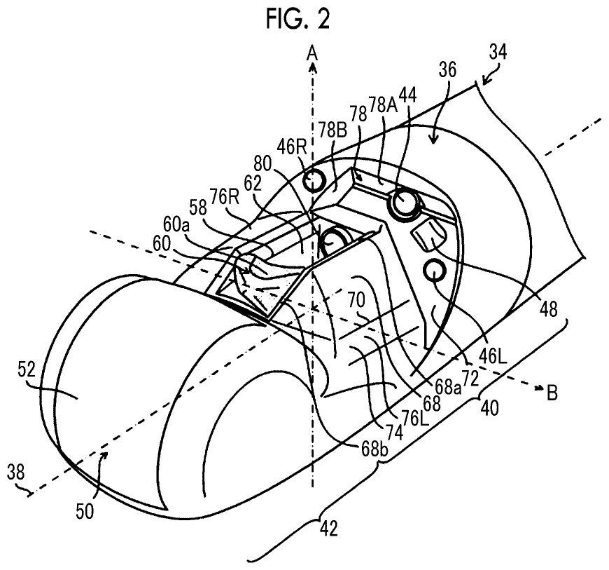

The ultrasound processor device 114 generates an ultrasound image based on an output signal of an ultrasonic transducer (see FIG. 2) 50 provided in the distal end rigid portion 34 (an echo signal reflected from an observation target part from which ultrasound is emitted) and displays the ultrasound image.

The endoscope processor device 116 includes a processor that receives and acquires an image signal (an image signal output from an imaging element 441 described below) acquired from an observation target part illuminated with illumination light from the light source device 118, performs various types of processing on the acquired image signal to generate an optical image, and displays the optical image on the monitor 120.

The processor may be configured by one or a plurality of pieces of hardware, and the type of hardware is not limited. For example, the processor may be configured by hardware such as a central processing unit (CPU), a micro processing unit (MPU), a programmable logic device such as a field-programmable gate array (FPGA), a dedicated circuit for executing specific processing such as an application-specific integrated circuit (ASIC), a graphics processing unit (GPU), or a neural processing unit (NPU). In addition, the processor has each unit or each means that executes various types of processing in the present embodiment. Types of hardware may be a combination of different types of hardware. In a case where a plurality of hardware are configured to execute one or a plurality of processes of a certain processor, the plurality of hardware may be present in devices physically separate from each other, or may be present in the same device. In addition, in any of the embodiments, the order of each processing by the processor is not limited to the above order and may be changed as appropriate. The hardware is composed of an electric circuit (circuitry) in which circuit elements such as semiconductor elements are combined.

In the example of FIG. 1, the ultrasound processor device 114 and the endoscope processor device 116 are configured with two devices (computers) provided separately. However, the present invention is not limited thereto, and both the ultrasound processor device 114 and the endoscope processor device 116 may be configured by one device.

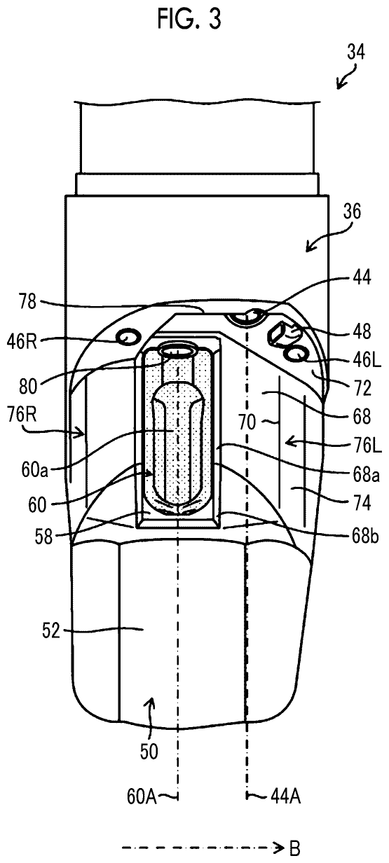

The light source device 118 generates illumination light such as white light consisting of three primary colors of red light, green light, and blue light, or specific wavelength light, and propagates the illumination light through a light guide (not shown) or the like in the ultrasonic endoscope 1 to emit the illumination light from illumination windows 46R and 46L (see FIGS. 2 and 3) to illuminate the observation target part in the body cavity in order to image the observation target part in the body cavity using an observation window 44 (see FIGS. 2 and 3) and acquire the image signal.

The monitor 120 displays the ultrasound image and the optical image generated by the ultrasound processor device 114 and the endoscope processor device 116. In regard to the display of the ultrasound image and the optical image, only one of these images may be switched and displayed as appropriate on the monitor 120, or both images may be displayed simultaneously.

In the present embodiment, the ultrasound image and the optical image are displayed on one monitor 120, but a monitor for displaying the ultrasound image and a monitor for displaying the optical image may be separately provided. In addition, the ultrasound image and the optical image may be displayed in a display form other than the monitor 120, for example, in a form of being displayed on a display of a terminal carried by the operator.

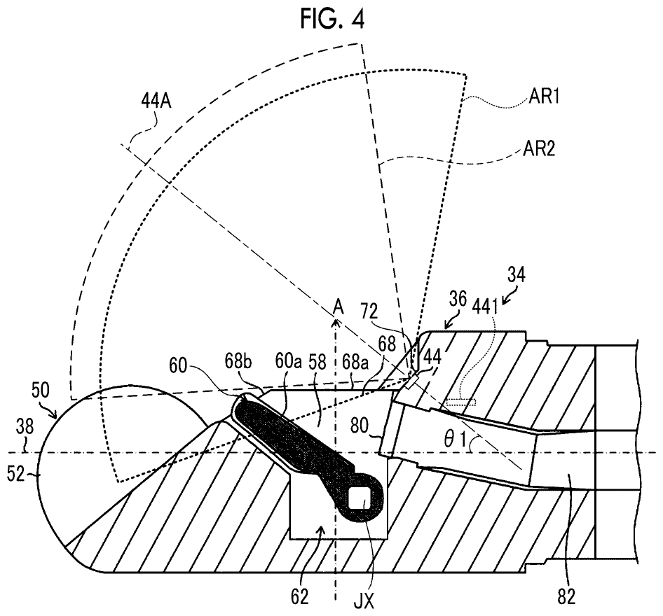

FIG. 2 is a perspective view showing an appearance of the distal end rigid portion 34. FIG. 3 is a plan view (top view) of the distal end rigid portion 34. FIG. 4 is a side cross-sectional view of the distal end rigid portion 34.

The distal end rigid portion 34 includes a distal end rigid portion body (frame body) 36 that forms an outer wall or an internal partition wall, and each component disposed in the distal end rigid portion 34 is accommodated and held in a housing portion defined by the distal end rigid portion body 36.

A part of the distal end rigid portion body 36 can be attachably and detachably removed as a separate block, and each component can be assembled to a predetermined housing portion in a state where the separate block is removed. After each component is assembled to the housing portion, the separate block is attached to the distal end rigid portion body 36, so that each component is accommodated and held in the housing portion and is fixed to the distal end rigid portion 34.

The distal end rigid portion body 36 is formed of an insulating material having an insulating property, for example, a resin material such as a methacrylic resin or a plastic including polycarbonate.

As shown in FIG. 2, the distal end rigid portion 34 is configured by a base part 40 that constitutes the distal end rigid portion body 36 and an extension part 42 that is extended from the base part 40 to the distal end side and that holds the ultrasonic transducer 50.

A convex type ultrasonic transducer 50 that has an ultrasonic wave transmission and reception surface 52 formed by arranging a plurality of transducers that transmit and receive ultrasound waves in a curved shape along an axial direction of the distal end rigid portion 34 is disposed in the extension part 42. The ultrasonic transducer 50 acquires data for generating an ultrasound image of a body tissue. Here, the axial direction of the distal end rigid portion 34 is synonymous with a longitudinal direction of the insertion part 12. The ultrasonic wave transmission and reception surface 52 constitutes a region in which the plurality of transducers are arranged.

The base part 40 is provided with the observation window 44, the illumination windows 46L and 46R, an air and water supply nozzle 48, an opening portion 58 that leads out a treatment tool, and a standing wall portion 68 provided around the opening portion 58.

The opening portion 58 is provided at a center of an opening forming surface 70 provided on the extension part 42 side of the base part 40, and the treatment tool is led out from the opening portion 58 to a scanning range of the ultrasonic transducer 50. The opening portion 58 is provided on the opening forming surface 70 and is formed in a first direction perpendicular to an axis 38 of the distal end rigid portion 34, on one side of an elevator housing portion 62 provided in the distal end rigid portion body 36. The opening portion 58 can also be formed in a direction having a component toward one side of the first direction and a component toward the distal end side of the axial direction of the distal end rigid portion 34 as an opening direction. That is, the opening portion 58 can also be formed to open in an upward direction (one side of the first direction) toward the distal end of the distal end rigid portion 34 in the opening direction. Here, the opening direction refers to a normal direction of a surface surrounded by an edge portion of the opening portion 58.

In the present specification, “one side of the first direction” refers to a direction perpendicular to the axis 38 of the distal end rigid portion 34, as indicated by an arrow A in FIG. 4, in which the opening portion 58 of the elevator housing portion 62 is formed. In addition, as shown in FIG. 2, a “second direction” refers to a direction shown with an arrow B, which is perpendicular to both the axial direction of the distal end rigid portion body 34 and the first direction shown with the arrow A. The direction indicated by the arrow B is a direction in which the plurality of transducers are arranged on the ultrasonic wave transmission and reception surface 52. In the present specification, the one side in the first direction will be referred to as “up” and “upward”, and the other side in the first direction will be referred to as “down” and “downward” in some cases. In addition, one side of the second direction may be referred to as “right” and “right side”, and the other side of the second direction may be referred to as “left” and “left side”.

The treatment tool is inserted from the treatment tool inlet port 24 of the operating part 10. The opening forming surface 70 is defined by a surface whose normal vector has a component oriented toward the opening direction. The opening direction is oriented upward, and the opening forming surface 70 is provided parallel to the axis 38 of the distal end rigid portion 34. In addition, the opening forming surface 70 may be provided to be inclined downward toward the distal end side of the distal end rigid portion 34. The opening portion 58 refers to a portion from which the treatment tool is led out from the elevator housing portion 62 by an elevator 60 described below.

By setting the position of the opening portion 58 to the proximal end side of the ultrasonic transducer 50 and setting the position to the distal end side of an observation means forming surface 72 as described below, the distance between the ultrasonic transducer 50 and the opening portion 58 can be reduced. Therefore, the distance from the position immediately after being led out from the opening portion 58 to the treatment position by the treatment tool can be reduced, so that the lateral deflection of the treatment tool can be reduced, and the treatment tool can be inserted into a target position.

The standing wall portion 68 is provided around the opening portion 58. The standing wall portion 68 can prevent the lateral deflection of the treatment tool led out from the opening portion 58, and the treatment tool can stably perform the treatment at the target position. The standing wall portion 68 may be formed as long as it can prevent the lateral deflection of the treatment tool, and it is not necessary to provide the standing wall portion 68 around the entire periphery of the opening portion 58. Specifically, it is preferable that the standing wall portion 68 is formed to rise upward from both sides of the opening portion 58 in a direction in which the treatment tool is led out. In the example of the drawing, the standing wall portion 68 is provided on the entire surface of the opening forming surface 70. In addition, the opening forming surface 70 and the standing wall portion 68 may be integrally formed.

The distal end rigid portion body 36 is provided with light guide retreat wall portions 76L and 76R that are cut obliquely downward at both left and right side portions of an opening forming surface portion 74 that forms the opening forming surface 70. As described above, by forming the light guide retreat wall portions 76L and 76R, it is possible to suppress blocking of the illumination light from the illumination windows 46L and 46R, and it is possible to prevent the occurrence of uneven illumination or the occurrence of shadowed portions. The light guide retreat wall portions 76L and 76R are not limited to being cut obliquely downward, and may be configured to be cut vertically or obliquely forward.

The observation window 44 is disposed on the observation means forming surface 72 provided on the proximal end side of the opening forming surface 70. An imaging system unit in which an imaging optical system and the imaging element 441 (see FIG. 4) are integrally assembled is accommodated inside the observation means forming surface 72. As a result, light from the subject is taken in from the observation window 44 and forms an image on the imaging element 441 through the imaging optical system, the subject is imaged, and the image signal is output from the imaging element 441. The observation means forming surface 72 is defined by a surface whose normal vector has a component oriented toward the distal end side of the distal end rigid portion 34 in the axial direction. The observation means forming surface 72 is formed as an inclined surface that is inclined upward toward the proximal end side of the distal end rigid portion body 36. By setting the observation means forming surface 72 to a surface whose normal vector has a component oriented toward the distal end side and by providing the observation window 44, it is possible to observe the treatment tool led out from the opening portion 58 through the observation window 44. The observation means forming surface 72 may be configured by a vertical surface perpendicular to the axial direction of the distal end rigid portion 34.

The illumination windows 46L and 46R are provided on both left and right sides of the observation means forming surface 72 with the observation window 44 interposed therebetween. A light emitting unit that constitutes an illumination unit is accommodated inside the observation means forming surface 72. The illumination light transmitted from the light source device 118 connected to the universal cord 14 through the light guide is emitted from the light emitting unit, and the illumination light is emitted to the subject in an imaging range of the imaging element 441 through the illumination windows 46L and 46R.

The air and water supply nozzle 48 is provided on the observation means forming surface 72, and a washing solution, water, air, or the like (hereinafter, also referred to as “washing solution or the like”) is jetted from the air and water supply nozzle 48 toward the observation window 44 by operating the air and water supply button 22 of the operating part 10, so that the observation window 44 is washed.

The observation means forming surface 72 has a deflection part 78 at a position facing the air and water supply nozzle 48 with the observation window 44 interposed therebetween. The deflection part 78 is disposed to protrude from the observation means forming surface 72. The deflection part 78 may be integrally formed with the observation means forming surface 72 or may be fixed separately. The washing solution or the like jetted from the air and water supply nozzle 48 toward the observation window 44 collides with the deflection part 78. The direction of the washing solution or the like that collides with the deflection part 78 is changed toward the opening portion 58 and is supplied to the opening portion 58. As a result, the washing or the like is performed in the opening portion 58.

The shape of the deflection part 78 is not particularly limited as long as the washing solution or the like that has passed through the observation window 44 can be directed toward the opening portion 58. As shown in FIGS. 2 and 3, the deflection part 78 may be configured by two surfaces 78A and 78B consisting of planes orthogonal to each other, or may be configured by a surface consisting of a curved shape such as an arc shape, an elliptical arc shape, or a parabolic shape.

The opening portion 58 is formed with a recessed elevator housing portion 62 that is connected to the opening portion 58, and a treatment tool outlet port 80 is disposed on the proximal end side of the recessed elevator housing portion 62.

The treatment tool outlet port 80 communicates with a treatment tool insertion channel 82 (see FIG. 4) inserted into the insertion part 12, and the treatment tool inserted from the treatment tool inlet port 24 (see FIG. 1) of the operating part 10 is led out from the treatment tool outlet port 80 to the elevator housing portion 62.

The elevator 60 is disposed at a position of the elevator housing portion 62 in front of the treatment tool outlet port 80. As shown in FIGS. 3 and 4, the entire elevator 60 is provided between the ultrasonic transducer 50 and the observation window 44 in the longitudinal direction of the insertion part 12. The elevator 60 is provided radially closer to a center (closer to an axis 38) of the insertion part 12 than the observation window 44. A part of the proximal end side of the elevator 60 may be disposed to overlap the observation window 44 in a side view of FIG. 4. The radial direction of the insertion part 12 means a direction orthogonal to the longitudinal direction of the insertion part 12.

The elevator 60 is formed of a metal material such as stainless steel, and has a concave guide surface 60a on an upper surface side, which curves upward from the proximal end side to the distal end side of the distal end rigid portion body 36. The treatment tool led out from the treatment tool outlet port 80 is curved upward with respect to the axial direction (longitudinal direction of the insertion part 12) of the distal end rigid portion 34 along the guide surface 60a and is led out to the outside from the opening portion 58 and the edge portion of the standing wall portion 68 on the upper side of the elevator housing portion 62.

The elevator 60 is rotated around a rotation shaft JX by operating an elevator control lever (not shown) provided on the operating part 10 to perform a standing operation upward, and the treatment tool led out from the treatment tool outlet port 80 can change an outlet direction (outlet angle) of the treatment tool by performing the standing operation of the elevator 60 to adjust an elevation angle from a lowered state. A position of the elevator 60 in the lowered state is referred to as a lowered position, and a position of the elevator 60 in a state where the elevator 60 is in a standing state (a state where a standing angle is greater than 0 degrees and equal to or less than an upper limit value) from the lowered state is referred to as a standing position.

The treatment tool insertion channel 82 is also connected to a suction channel (not shown), and by operating the suction button 20 of the operating part 10, suction of body fluid or the like from the opening portion 58 can also be performed.

In a case of observing the inside of the subject with the ultrasonic endoscope 1, the ultrasonic transducer 50 may be used by being pressed against a wall of a digestive tract or may be used by filling the digestive tract with water (sterile water filling method). In a case where an optical image is captured through the observation window 44 in water, due to a difference in refractive index between water and air, the imaging range of the imaging element 441 becomes narrower in water than in air.

FIG. 4 shows a first imaging range AR1 of the imaging element 441 in a case where the distal end rigid portion 34 is in air (synonymous with a case where the observation window 44 is in air), a second imaging range AR2 of the imaging element 441 in a case where the distal end rigid portion 34 is in water (synonymous with a case where the observation window 44 is in water), and an optical axis 44A of an objective lens included in the imaging optical system provided adjacent to the observation window 44 (synonymous with an optical axis of the observation window 44). The optical axis 44A extends in a direction intersecting the longitudinal direction of the insertion part 12.

The imaging range of the imaging element 441 is defined as an angle range centered on the optical axis 44A of the observation window 44 in a side view as viewed from the direction of the arrow B (a direction perpendicular to both the longitudinal direction of the insertion part 12 and the movement direction of the elevator 60). As shown in FIG. 4, the imaging range AR2 is included in the imaging range AR1 and is narrower than the imaging range AR1. For example, the first imaging range AR1 is an angle range of 100 degrees or more and 140 degrees or less, and the second imaging range AR2 is an angle range of 70 degrees or more and 90 degrees or less.

In the ultrasonic endoscope 1, at least a part of the elevator 60 in the lowered position and at least a part of the ultrasonic wave transmission and reception surface 52 are included in the first imaging range AR1, and the elevator 60 in the lowered position is not included in the second imaging range AR2, and at least a part of the ultrasonic wave transmission and reception surface 52 is included in the second imaging range AR2. Further, at least a part of the elevator 60 in the standing position may be included in the first imaging range AR1, and the elevator 60 in the standing position need not be included in the second imaging range AR2. The fact that the elevator 60 is not included in the imaging range means that the elevator 60 is not substantially included in the imaging range, allowing customary tolerances.

More specifically, at least the distal end of the elevator 60 in the lowered state is included in a range on a side close to the axis 38 of two angle ranges obtained by excluding the second imaging range AR2 from the first imaging range AR1. The ultrasonic wave transmission and reception surface 52 is configured to extend across both the range and the second imaging range AR2.

In a case where an angle of the first imaging range AR1 is denoted by θ2, an angle of the second imaging range AR2 is denoted by θ3, and an angle between the optical axis 44A and the axis 38 is denoted by θ1, the range on the side close to the axis 38 of the two angle ranges obtained by excluding the second imaging range AR2 from the first imaging range AR1 is defined as a range of (θ2−θ3)/2 on both sides with a straight line extending from the center of the observation window 44 and intersecting the axis 38 and forming an angle of (θ2+θ3)/4−θ1 with the axis 38 as a central axis.

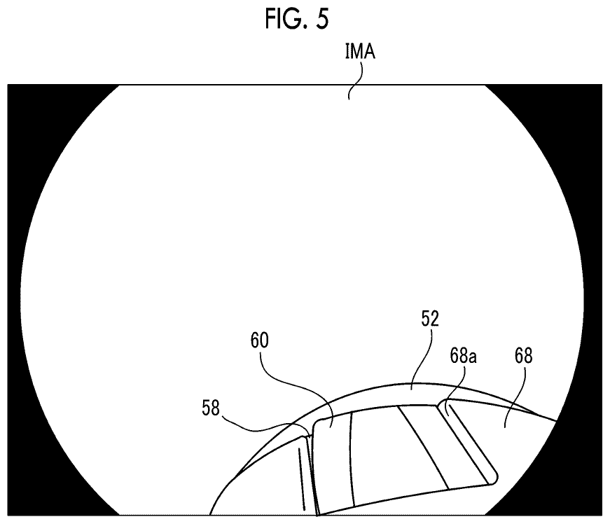

In a case where the imaging is performed by the imaging element 441 in a state where the elevator 60 is in the lowered state and the distal end rigid portion 34 is in air, the first optical image IMA obtained by processing the image signal output from the imaging element 441 is displayed on the monitor 120. FIG. 5 is a diagram showing an example of the first optical image IMA displayed on the monitor 120.

As shown in FIG. 5, the first optical image IMA includes a part of the ultrasonic wave transmission and reception surface 52 and a part of the elevator 60 in the lowered position. In a case where the entire elevator 60 is included in the first imaging range AR1, the first optical image IMA includes a part of the ultrasonic wave transmission and reception surface 52 and the entire elevator 60 in the lowered position. In a case where the elevator 60 in the standing position is included in the first imaging range AR1, the elevator 60 is included in the first optical image IMA even in a case where the elevator 60 is in the standing position.

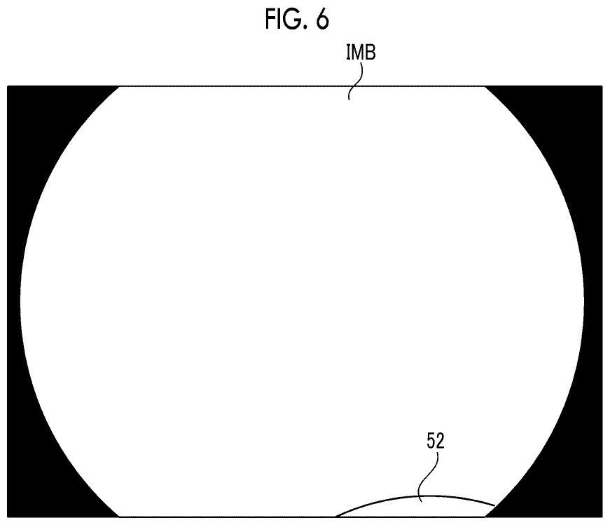

In a case where the imaging is performed by the imaging element 441 in a state where the elevator 60 is in the lowered state and the distal end rigid portion 34 is in water, a second optical image IMB obtained by processing the image signal output from the imaging element 441 is displayed on the monitor 120. FIG. 6 is a diagram showing an example of the second optical image IMB displayed on the monitor 120.

As shown in FIG. 6, the second optical image IMB includes a part of the ultrasonic wave transmission and reception surface 52, but does not include the elevator 60 in the lowered position. In a case where the elevator 60 in the standing position is not included in the second imaging range AR2, the elevator 60 is not included in the second optical image IMB even in a case where the elevator 60 is in the standing position.

In a case where the ultrasonic endoscope 1 is used in water, the treatment tool is not used, so that the elevator 60 is held in the lowered state. Therefore, as shown in FIG. 6, the influence on the operation of the ultrasonic endoscope 1 is not affected even in a case where the elevator 60 is not included in the displayed optical image. On the contrary, by not including the elevator 60 in the optical image, the observation range can be widened, and the operability of the ultrasonic endoscope 1 can be improved.

On the other hand, in a case where the ultrasonic endoscope 1 is used in air, the treatment tool may be used. Therefore, as shown in FIG. 5, by including the elevator 60 in the displayed optical image, the treatment using the treatment tool can be accurately and safely performed.

In a case where the ultrasonic endoscope 1 is used in water, the ultrasound image may be acquired. Therefore, as shown in FIG. 6, by including a part or all of the ultrasonic wave transmission and reception surface 52 in the displayed optical image, the positioning of the ultrasonic wave transmission and reception surface 52 can be easily performed, and the operability can be improved.

It is not essential that at least a part of the ultrasonic wave transmission and reception surface 52 is included in the second imaging range AR2, and the ultrasonic wave transmission and reception surface 52 need not be included in the second imaging range AR2. The fact that the ultrasonic wave transmission and reception surface 52 is not included in the imaging range means that the ultrasonic wave transmission and reception surface 52 is not substantially included in the imaging range, allowing customary tolerances.

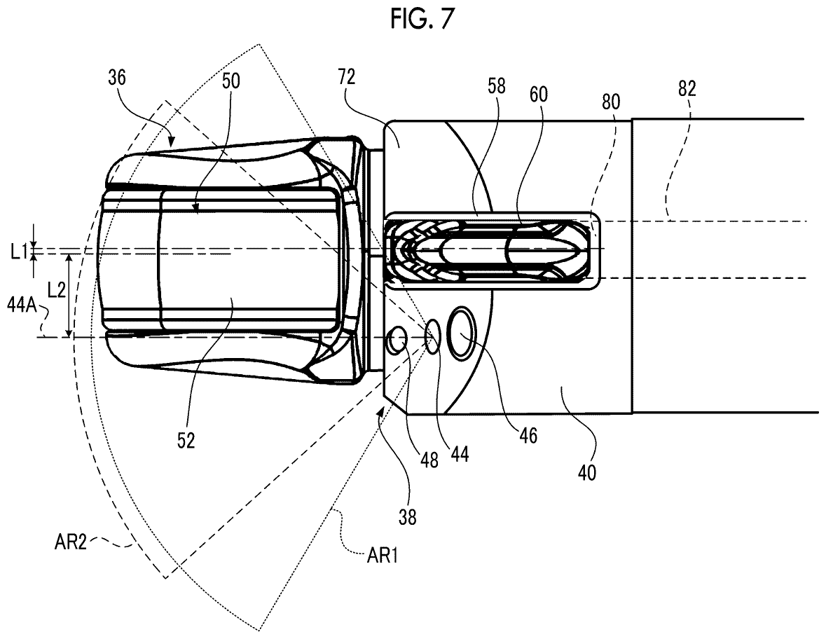

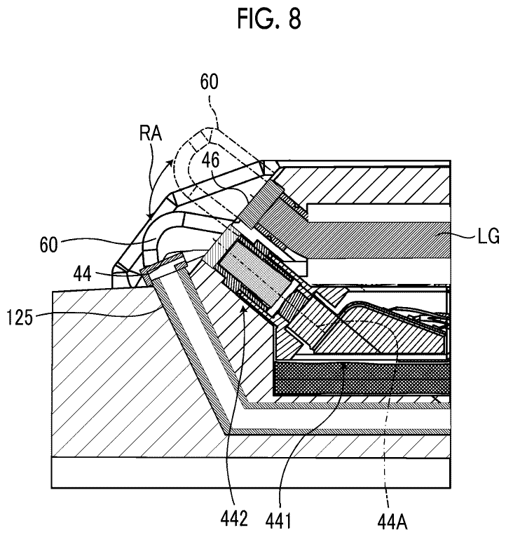

FIG. 7 is a plan view showing a modification example of the distal end rigid portion 34. FIG. 8 is a side cross-sectional view of the distal end rigid portion 34 shown in FIG. 7. The distal end rigid portion 34 shown in FIG. 7 is different from the configuration shown in FIG. 2 in that the position of the elevator 60 and the opening portion 58 accommodating the elevator 60 is moved to the proximal end side and the illumination windows 46R and 46L are changed to the illumination window 46.

The air and water supply nozzle 48 is connected to an air and water supply hole 125. The observation window 44 is disposed in front of an imaging unit including an imaging optical system 442 and the imaging element 441. A light guide LG is connected to the illumination window 46. The elevator 60 is configured to rotate within a predetermined rotation range RA.

As shown in FIG. 8, in the distal end rigid portion 34 of the modification example, the elevator 60 is provided at a position overlapping the observation window 44 in a side view as viewed from a direction perpendicular to both the longitudinal direction of the insertion part 12 and the movement direction of the elevator 60. The observation window 44 and the elevator 60 overlap each other in a side view in any of a state where the elevator 60 is in the lowered position and a state where the elevator 60 is in the standing position. In a plan view shown in FIG. 7, the elevator 60 is provided with a rotation shaft at an end part on the proximal end side, and a rotation center of the elevator 60 is present on the proximal end side with respect to the observation window 44. In addition, a distal end part of the elevator 60 is positioned between the observation window 44 and the ultrasonic transducer 50.

FIG. 7 shows a distance L1 between a first center of the ultrasonic wave transmission and reception surface 52 in a left-right direction and a second center of a region on which the treatment tool is placed in the left-right direction of the elevator 60. In addition, FIG. 7 shows a distance L2 between a third center of the observation window 44 in the left-right direction and the first center. The distance L1 is smaller than the distance L2.

With such a configuration, in a case where the ultrasonic endoscope 1 is used in air, the ultrasonic wave transmission and reception surface 52 and the elevator 60 can be positioned on one end side in a horizontal direction in the optical image displayed on the monitor 120, and the treatment tool can be operated satisfactorily while ensuring a wide field of view.

In the modification example shown in FIG. 7, in a plan view as viewed in the movement direction of the elevator 60, at least a part or all of the elevator 60 in the lowered position is included in the first imaging range AR1, and the elevator 60 in the lowered position is not included in the second imaging range AR2. Even in this modification example, at least a part or all of the elevator 60 in the standing position may be included in the first imaging range AR1, and the elevator 60 in the standing position need not be included in the second imaging range AR2. In addition, at least one of the ultrasonic wave transmission and reception surfaces 52 may be included in the second imaging range AR2.

In the modification example shown in FIG. 7, in a case where the imaging is performed by the imaging element 441 in a state where the elevator 60 is in the lowered state and the distal end rigid portion 34 is in air, a first optical image IMC obtained by processing the image signal output from the imaging element 441 is displayed on the monitor 120.

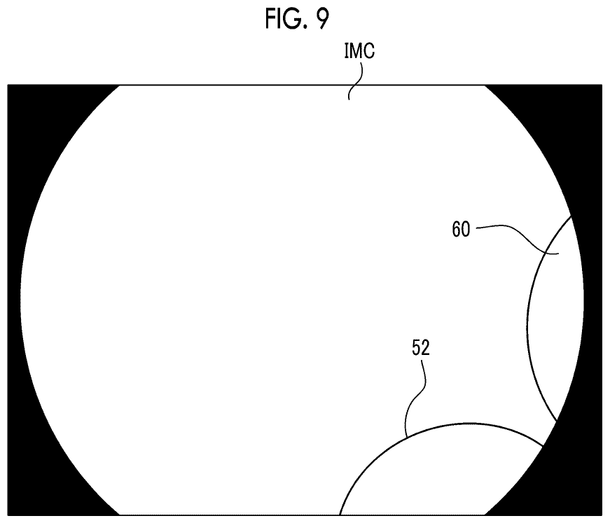

FIG. 9 is a diagram showing an example of the first optical image IMC displayed on the monitor 120. As shown in FIG. 9, the first optical image IMC includes a part of the ultrasonic wave transmission and reception surface 52 and a part of the elevator 60 in the lowered position. In a case where the entire elevator 60 is included in the first imaging range AR1, the first optical image IMC includes a part of the ultrasonic wave transmission and reception surface 52 and the entire elevator 60 in the lowered position. In a case where the elevator 60 in the standing position is included in the first imaging range AR1, the elevator 60 is included in the first optical image IMC even in a case where the elevator 60 is in the standing position. As shown in FIG. 9, the elevator 60 included in the first optical image IMC is displayed to overlap an end edge on one side of the first optical image IMC in the horizontal direction.

In the modification example shown in FIG. 7, in a case where the imaging is performed by the imaging element 441 in a state where the elevator 60 is in the lowered state and the distal end rigid portion 34 is in water, a second optical image IMD obtained by processing the image signal output from the imaging element 441 is displayed on the monitor 120.

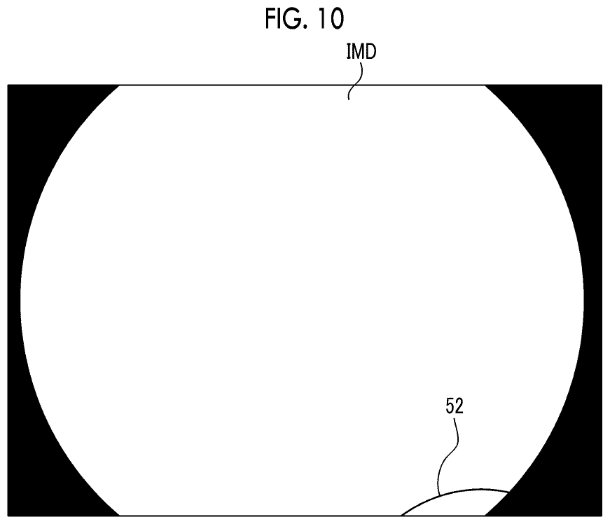

FIG. 10 is a diagram showing an example of the second optical image IMD displayed on the monitor 120. As shown in FIG. 10, the second optical image IMD includes a part of the ultrasonic wave transmission and reception surface 52, but does not include the elevator 60 in the lowered position. In a case where the elevator 60 in the standing position is not included in the second imaging range AR2, the elevator 60 is not included in the second optical image IMD even in a case where the elevator 60 is in the standing position.

As described above, with the distal end rigid portion 34 of the modification example, in a case where the ultrasonic endoscope 1 is used in water, a region hidden by the elevator 60 in the displayed optical image can be eliminated, and the operability can be improved.

In the above description, at least a part of the elevator 60 is included in the first imaging range AR1, and the elevator 60 is not included in the second imaging range AR2. However, at least a part of the elevator 60 may be included in the first imaging range AR1, and the elevator 60 may be included in the second imaging range AR2. However, in this case, in a case where the optical image is displayed on the monitor 120, a display range is narrowed in a case of being used in water.

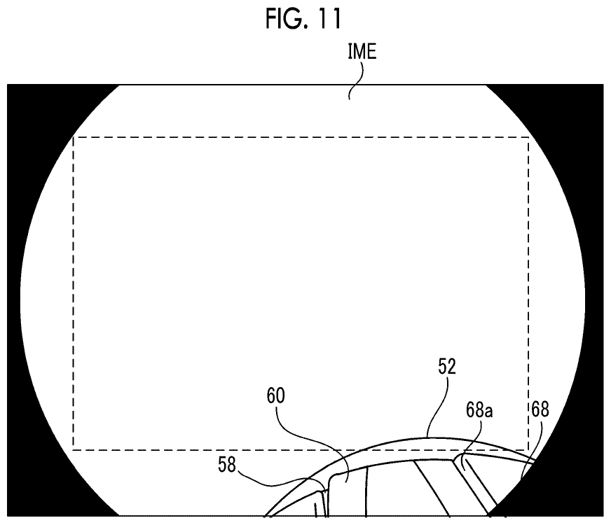

By way of example, a case will be described in which, in a case where the ultrasound endoscope 1 is used in water, imaging of a subject region corresponding to an optical image IME shown in FIG. 11 is possible. In this case, in a case where the ultrasonic endoscope 1 is used in water, by cutting out and enlarging the range shown by a broken line frame in FIG. 11, an image corresponding to the second optical image IMB shown in FIG. 6 can be displayed.

It is preferable that the angle range corresponding to the angle of view of the optical image displayed in a case where the observation window 44 is in air is an angle range of 100 degrees or more and 140 degrees or less. It is preferable that the angle range corresponding to the angle of view of the optical image displayed in a case where the observation window 44 is in water is an angle range of 70 degrees or more and 90 degrees or less.

As described above, at least the following matters are described in the present specification.
(1)

An ultrasound endoscope system comprising:
an insertion part that is inserted into a subject;
a plurality of transducers that are provided on a distal end side of the insertion part and that transmit and receive ultrasound waves;
an observation window that is provided on a proximal end side of the plurality of transducers in the insertion part;
an imaging element that images the subject through the observation window and outputs an image signal of the subject;
a treatment tool outlet port that is provided on the distal end side of the insertion part and that is an outlet port for a treatment tool;
an elevator that is provided at least partially between the plurality of transducers and the observation window in a longitudinal direction of the insertion part and that is able to change an outlet direction of the treatment tool led out from the outlet port; and
a processor that acquires the image signal output from the imaging element,
in which the processor is configured to display a first optical image generated from the image signal on a display device in a case where the observation window is in air, and display a second optical image generated from the image signal on the display device in a case where the observation window is in water,
at least a part of the elevator is included in the first optical image, and
the elevator is not included in the second optical image.
(2)

The ultrasound endoscope system according to (1),
in which at least a part of the elevator in a lowered position is included in the first optical image, and
the elevator in the lowered position is not included in the second optical image.
(3)

The ultrasound endoscope system according to (2),
in which at least a part of the elevator in a standing position is included in the first optical image, and
the elevator in the standing position is not included in the second optical image.
(4)

The ultrasound endoscope system according to any one of (1) to (3),
in which at least a part of a region in which the plurality of transducers are arranged is included in the second optical image.
(5)

The ultrasound endoscope system according to any one of (1) to (4),
in which at least a part of the elevator is included in a first imaging range of the imaging element in a case where the observation window is in air, and
the elevator is not included in a second imaging range of the imaging element in a case where the observation window is in water.
(6)

The ultrasound endoscope system according to (5),
in which the second imaging range is included in the first imaging range.
(7)

The ultrasound endoscope system according to (5) or (6),
in which the first imaging range is an angle range of 100 degrees or more and 140 degrees or less, and
the second imaging range is an angle range of 70 degrees or more and 90 degrees or less.
(8)

The ultrasound endoscope system according to any one of (1) to (7),
in which an optical axis of the observation window intersects the longitudinal direction of the insertion part.
(9)

The ultrasound endoscope system according to any one of (1) to (8),
in which the elevator is provided closer to the distal end side than the observation window.
(10)

The ultrasound endoscope system according to any one of (1) to (9),
in which the elevator is provided radially closer to a center of the insertion part than the observation window.
(11)

The ultrasound endoscope system according to any one of (1) to (8),
in which the elevator is provided at a position overlapping the observation window in a side view as viewed from a direction perpendicular to both the longitudinal direction of the insertion part and a movement direction of the elevator.
(12)

The ultrasound endoscope system according to (11),
in which the elevator in a lowered position is provided at a position overlapping the observation window in the side view.
(13)

The ultrasound endoscope system according to (11) or (12),
in which, in the side view, a rotation center of the elevator is on a proximal end side with respect to the observation window.
(14)

The ultrasound endoscope system according to any one of (11) to (13),
in which, in a plan view as viewed from the movement direction of the elevator, a distance between a first center of a region in which the plurality of transducers are arranged in a direction perpendicular to the longitudinal direction of the insertion part and a second center of a region of the elevator on which the treatment tool is placed in the direction perpendicular to the longitudinal direction of the insertion part is smaller than a distance between a third center of the observation window in the direction perpendicular to the longitudinal direction of the insertion part and the first center.

According to the configuration of (14), since the plurality of transducers and the elevator, which are large components of the ultrasonic endoscope, are disposed on the center side of the endoscope and the observation window is disposed on the end side of the endoscope, the insertion part can be made thin.
(15)

The ultrasound endoscope system according to any one of (11) to (14),
in which the elevator included in the first optical image overlaps an end edge on one side of the first optical image in a horizontal direction.
(16)

The ultrasound endoscope system according to any one of (11) to (15),
in which at least a part of a region in which the plurality of transducers are arranged is included in a first imaging range of the imaging element in a case where the observation window is in air, and
the region in which the plurality of transducers are arranged is not included in a second imaging range of the imaging element in a case where the observation window is in water.
(17)

The ultrasound endoscope system according to any one of (11) to (16),
in which at least a part of a region in which the plurality of transducers are arranged is included in the first optical image, and
the region in which the plurality of transducers are arranged is not included in the second optical image.

### EXPLANATION OF REFERENCES

1: ultrasonic endoscope
10: operating part
12: insertion part
14: universal cord
16: angle knob
20: suction button
22: air and water supply button
24: treatment tool inlet port
30: soft part
32: bendable part
34: distal end rigid portion
36: distal end rigid portion body
38: axis
40: base part
42: extension part
44: observation window
44A: optical axis
46, 46L, 46R: illumination window
48: air and water supply nozzle
50: ultrasonic transducer
52: ultrasonic wave transmission and reception surface
58: opening portion
60: elevator
60a: guide surface
62: elevator housing portion
68: standing wall portion
70: opening forming surface
72: observation means forming surface
74: opening forming surface portion
76L: light guide retreat wall portion
78: deflection part
78A: surface
80: treatment tool outlet port
82: treatment tool insertion channel
100: ultrasound endoscope system
114: ultrasound processor device
116: endoscope processor device
118: light source device
120: monitor
121a: water supply tank
121b: suction pump
125: air and water supply hole
132a, 132b, 132c: connector
134a: air and water supply tube
134b: suction tube
441: imaging element
442: imaging optical system
AR1: first imaging range
AR2: second imaging range
L1, L2: distance
JX: rotation shaft

What is claimed is:

1. An ultrasound endoscope system comprising:
an insertion part that is inserted into a subject;
a plurality of transducers that are provided on a distal end side of the insertion part and that transmit and receive ultrasound waves;
an observation window that is provided on a proximal end side of the plurality of transducers in the insertion part;
an imaging element that images the subject through the observation window and that outputs an image signal of the subject;
a treatment tool outlet port that is provided on the distal end side of the insertion part and that is an outlet port for a treatment tool;
an elevator that is provided at least partially between the plurality of transducers and the observation window in a longitudinal direction of the insertion part and that is able to change an outlet direction of the treatment tool led out from the outlet port; and
a processor that acquires the image signal output from the imaging element,
wherein the processor is configured to display a first optical image generated from the image signal on a display device in a case where the observation window is in air, and display a second optical image generated from the image signal on the display device in a case where the observation window is in water,
at least a part of the elevator is included in the first optical image, and
the elevator is not included in the second optical image.

2. The ultrasound endoscope system according to claim 1,
wherein at least a part of the elevator in a lowered position is included in the first optical image, and
the elevator in the lowered position is not included in the second optical image.

3. The ultrasound endoscope system according to claim 2,
wherein at least a part of the elevator in a standing position is included in the first optical image, and
the elevator in the standing position is not included in the second optical image.

4. The ultrasound endoscope system according to claim 1,
wherein at least a part of a region in which the plurality of transducers are arranged is included in the second optical image.

5. The ultrasound endoscope system according to claim 1,
wherein at least a part of the elevator is included in a first imaging range of the imaging element in a case where the observation window is in air, and
the elevator is not included in a second imaging range of the imaging element in a case where the observation window is in water.

6. The ultrasound endoscope system according to claim 5,
wherein the second imaging range is included in the first imaging range.

7. The ultrasound endoscope system according to claim 5,
wherein the first imaging range is an angle range of 100 degrees or more and 140 degrees or less, and
the second imaging range is an angle range of 70 degrees or more and 90 degrees or less.

8. The ultrasound endoscope system according to claim 5,
wherein an optical axis of the observation window intersects the longitudinal direction of the insertion part.

9. The ultrasound endoscope system according to claim 1,
wherein the elevator is provided closer to the distal end side than the observation window.

10. The ultrasound endoscope system according to claim 1,
wherein the elevator is provided radially closer to a center of the insertion part than the observation window.

11. The ultrasound endoscope system according to claim 1,
wherein the elevator is provided at a position overlapping the observation window in a side view as viewed from a direction perpendicular to both the longitudinal direction of the insertion part and a movement direction of the elevator.

12. The ultrasound endoscope system according to claim 11,
wherein the elevator in a lowered position is provided at a position overlapping the observation window in the side view.

13. The ultrasound endoscope system according to claim 11,
wherein, in the side view, a rotation center of the elevator is on a proximal end side with respect to the observation window.

14. The ultrasound endoscope system according to claim 11,
wherein, in a plan view as viewed from the movement direction of the elevator, a distance between a first center of a region in which the plurality of transducers are arranged in a direction perpendicular to the longitudinal direction of the insertion part and a second center of a region of the elevator on which the treatment tool is placed in the direction perpendicular to the longitudinal direction of the insertion part is smaller than a distance between a third center of the observation window in the direction perpendicular to the longitudinal direction of the insertion part and the first center.

15. The ultrasound endoscope system according to claim 11,
wherein the elevator included in the first optical image overlaps an end edge on one side of the first optical image in a horizontal direction.

16. The ultrasound endoscope system according to claim 11,
wherein at least a part of a region in which the plurality of transducers are arranged is included in a first imaging range of the imaging element in a case where the observation window is in air, and
the region in which the plurality of transducers are arranged is not included in a second imaging range of the imaging element in a case where the observation window is in water.

17. The ultrasound endoscope system according to claim 11,
wherein at least a part of a region in which the plurality of transducers are arranged is included in the first optical image, and
the region in which the plurality of transducers are arranged is not included in the second optical image.
